(19) United States
(12) Patent Application Publication
(10) Pub. No.: US 2026/0062370 A1
(43) Pub. Date: Mar. 5, 2026

(54) METHODS AND SYSTEMS FOR PRODUCING BIOFUEL

(71) Applicant: Edgewood Renewables, LLC, Las Vegas, NV (US)

(72) Inventors: Daniel Evan BROWN, Las Vegas, NV (US); W. Stephen Patrick HARRINGTON, Washington, DC (US)

(21) Appl. No.: 19/311,699

(22) Filed: Aug. 27, 2025

Related U.S. Application Data
(60) Provisional application No. 63/689,436, filed on Aug. 30, 2024.

(51) Int. Cl. C07C 29/151; B01D 53/047; C01B 3/40; C01B 3/48; C01B 3/56; C10G 3/00

(52) U.S. Cl. CPC C07C 29/1518; B01D 53/047; C01B 3/40; C01B 3/48; C01B 3/56; C10G 3/50; B01D 2256/16; B01D 2257/502; B01D 2258/05; C01B 2203/0233; C01B 2203/042; C01B 2203/047; C01B 2203/061; C01B 2203/1058; C01B 2203/1241; C01B 2203/14; C10G 2300/1014; C10G 2300/1018; C10G 2400/04; C10G 2400/08

(57) ABSTRACT

A method of producing methanol includes supplying biomass feedstock to a hydrotreating unit to produce a biofuel and a biogenic off gas. The biogenic off gas is delivered to a methane reforming unit to generate a syngas having higher hydrogen content than the biogenic off gas. The method also includes delivering a first portion of the syngas to a hydrogen purification unit to produce a hydrogen stream. The hydrogen stream is delivered to the hydrotreating unit. A second portion of the syngas is delivered to a methanol generation unit. Methanol is generated from the second portion of the syngas by the methanol generation unit.

### CROSS-REFERENCE TO RELATED APPLICATION

This application claims the benefit of priority of U.S. Provisional Patent Application No. 63/689,436, filed on Aug. 30, 2024, which application is incorporated herein by reference in its entirety.

### BACKGROUND

### Field

Embodiments described herein are generally related to the production of biomethanol. In particular, embodiments of the present disclosure relate to the production of biomethanol at a biofuel facility.

### Description of the Related Art

One of the ways the global shipping industry is moving to reduce its carbon footprint is by using renewable methanol-powered ships. There are two types of renewable methanols: eMethanol and biomethanol.

Biomethanol is produced using the same process as the traditional methanol production process, but the biomethanol is made from a biogas feedstock instead of petroleum natural gas. Biogas is comprised primarily of CH4, CO2, CO, and H2, where all of the carbon is from a biogenic source (i.e., not petroleum based).

Sourcing the biogas can be a challenge. Ways to produce biogas include anaerobic digestion or gasification of biomass feedstocks. Anaerobic digestion is where microorganisms break down organic matter in the absence of oxygen and is typically applied to dairy manure or to landfill gas. Gasification is the process of thermally breaking down biomass, such as woody biomass, crop residues, or other agricultural waste.

However, there is a continuing need to efficiently source biogas for producing biomethanol.

### SUMMARY

In one embodiment, a method of producing methanol includes supplying biomass feedstock to a hydrotreating unit to produce a biofuel and a biogenic off gas. The biogenic off gas is delivered to a methane reforming unit to generate a syngas having higher hydrogen content than the biogenic off gas. The method also includes delivering a first portion of the syngas to a hydrogen purification unit to produce a hydrogen stream. The hydrogen stream is delivered to the hydrotreating unit. A second portion of the syngas is delivered to a methanol generation unit. Methanol is generated from the second portion of the syngas by the methanol generation unit.

In another embodiment, a method of producing biofuels includes supplying biomass feedstock to a hydrotreating unit to produce a first biofuel and a first biogenic off gas. The first biogenic off gas is delivered to a pre-reforming unit to produce a second biogenic off gas having a higher methane content than the first biogenic off gas. The second biogenic off gas is delivered to a methane reforming unit to generate a syngas having higher hydrogen content than the second biogenic off gas. A first portion of the syngas is delivered to a pressure swing unit. A second portion of the syngas is delivered to a methanol generation unit, and a second biofuel is generated from the second portion of the syngas. Hydrogen from the methanol generation unit is delivered to the pressure swing unit or the hydrotreating unit.

In another embodiment, a method of producing biofuels includes supplying biomass feedstock to a hydrotreating unit to produce a first biofuel and a first biogenic off gas. The method also includes pre-reforming the first biogenic off gas to produce a second biogenic off gas having a higher methane content than the first biogenic off gas. The second biogenic off gas is reformed to generate a syngas having a higher hydrogen content than the second biogenic off gas. Hydrogen is separated from a first portion of the syngas using a hydrogen purification unit. The separated hydrogen is delivered to the hydrotreating unit. A second portion of the syngas is used to generate a second biofuel.

In another embodiment, a method of producing methanol includes supplying biomass feedstock to a hydrotreating unit to produce a biofuel and a biogenic off gas. The method also includes delivering the biogenic off gas to a methane reforming unit to generate a syngas having higher hydrogen content than the biogenic off gas. The syngas is delivered to a hydrogen purification unit to produce a hydrogen stream and a gas stream. A first portion of the hydrogen stream is delivered to the hydrotreating unit, and a second portion of the hydrogen stream is delivered to a methanol generation unit. Methanol is generated using the methanol generation unit.

### BRIEF DESCRIPTION OF THE DRAWINGS

So that the manner in which the above recited features can be understood in detail, a more particular description of the disclosure, briefly summarized above, may be had by reference to embodiments, some of which are illustrated in the appended drawings. It is to be noted, however, that the appended drawings illustrate only typical embodiments and are therefore not to be considered limiting of its scope, for the disclosure may admit to other equally effective embodiments.

FIG. 1 shows an exemplary embodiment of a biofuel generation facility for making biofuel, according to some embodiments.

FIG. 2 shows another exemplary embodiment of a biofuel generation facility for making biofuel, according to some embodiments.

FIG. 3 shows another exemplary embodiment of a biofuel generation facility for making biofuel, according to some embodiments.

### DETAILED DESCRIPTION

Embodiments of the present disclosure provides for the production of biomethanol at a biofuel facility that makes biofuel such as renewable diesel (“RD”) or sustainable aviation fuel (“SAF”). In some embodiments, a methanol loop is added to a biofuel facility to allow for the production of biomethanol. The methanol loop advantageously allows the production of more biofuels per unit of feedstock, thereby increasing efficiency.

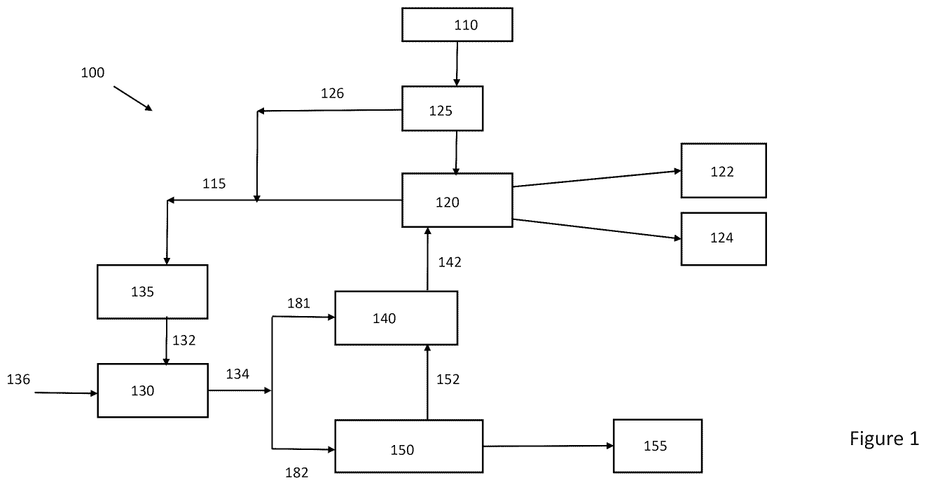

Embodiments of the present disclosure provide a method of producing biofuel, such as renewable diesel (“RD”) and a sustainable aviation fuel (“SAF”), and biomethanol. FIG. 1 shows an exemplary embodiment of a biofuel generation facility 100 for making biofuel. In this embodiment, the biofuel facility 100 includes a feedstock source 110, a hydrotreating unit 120, an optional pre-reforming unit 135, a steam methane reforming unit 130, a pressure swing adsorption unit 140, and a methanol generation unit 150. In some embodiments, the biofuel facility 100 includes an optional feedstock pretreatment unit 125 for processing the feedstock prior to sending the feedstock to the hydrotreating unit 120.

In one embodiment, the renewable diesel and the sustainable aviation fuel are made by hydroprocessing biomass such as esters and fatty acids (“HEFA”) from the feedstock source 110. Generally, the biomass includes agricultural oils and animal fats. Exemplary biomass includes renewable biomass such as distillers corn oil, beef tallow, used cooking oil, soybean oil, jatropha oil, castor oil, tall oil or tall oil fatty acids, canola, algae, Salicornia, palm oil, or other suitable triglycerides and fatty acids.

In some embodiments, the biomass feedstock undergoes a pretreatment process before being delivered to the hydrotreating unit 120. In one embodiment, the biomass is delivered to the feedstock pretreatment unit 125 where the biomass feedstock is thermally pretreated. The biomass feedstock is heated to a temperature from 600° F. to 850° F. or from 650° F. to 800° F. at a pressure from 300 psi to 500 psi. Water is supplied to hydrate the biomass feedstock. In some examples, the water is supplied in liquid form. The pretreatment process facilitates removal of impurities, including chlorides, metals such as calcium and magnesium, and gums (e.g., phosphorus), in the biomass feedstock.

The pretreatment process may produce a biogenic off gas 126, which is a type of biogas that can be used to produce methanol, as will be described below. The biogenic off gas 126 includes carbon monoxide and carbon dioxide as the largest components. The biogenic off gas 126 may also include one or more of methane, ethane, propane, and hydrogen. In some embodiments, at least a portion of the off gas 126 is delivered to the pre-reforming unit 135 or the steam methane reforming unit 130. The portion delivered to the pre-reforming unit 135 or the steam methane reforming unit 130 may be an amount from 70% to 100% or from 90% to 100% of the off gas 126. In some examples, a portion of the biogenic off gas 126 from the pretreatment process may be delivered to the hydrotreating unit 120 along with the pretreated biomass feedstock. Although a thermal pretreatment is disclosed, the biomass feedstock may be pretreated using other suitable pretreatment processes, such as bleaching earth and acid degumming.

The pretreated biomass feedstock is delivered to the hydrotreating unit 120 for hydroprocessing. In some embodiments, the biomass feedstock is delivered from the feedstock source 110 to the hydrotreating unit 120. Hydroprocessing involves adding hydrogen to the triglycerides or fatty acids biomass. Hydroprocessing may occur at a temperature from 550° F. to 750° F. and a pressure from 1100 psi to 1400 psi in the presence of a catalyst containing Ni, Mo, CoMo, NiMo, or combinations thereof. The hydroprocessing removes the oxygen from the biomass, e.g., triglycerides or the fatty acids. The removed oxygen is then converted to water or carbon dioxide. The hydroprocessing also saturates any unsaturated chains in the biomass. The hydroprocessing produces a saturated paraffin compound that can be further processed to produce either renewable diesel 122, sustainable aviation fuel 124, or both.

In some embodiments, the paraffin compounds are isomerized. For example, long-chain n-paraffins are isomerized into iso-paraffins of the same length. Isomerization beneficially improves the flow property of the renewable diesel fuel in cold weather by preventing freezing. In some embodiments, the paraffin compounds are cracked and isomerized to jet fuel chain length.

After isomerization, the paraffin compounds go through a fractionation process. The fractionation process separates the paraffin compounds into fractions suitable for biofuel use. In some examples, the fractions include a light fraction, a naphtha fraction, a middle distillate fraction, and a heavy fraction. In some examples, the light fraction may include hydrocarbons having a carbon chain length from 1 to 4. The light fraction may also include other gaseous components such as hydrogen and carbon monoxide, depending on the process from which the light fraction derives. The naphtha fraction includes a distilled hydrocarbon fraction, wherein the hydrocarbons have a carbon chain length of 5 to 10. The middle distillate includes a hydrocarbon fraction containing hydrocarbons having a carbon chain length of 11 to 20. The middle distillate is more desirable for use as a hydrocarbon biofuel. The heavy fraction includes a hydrocarbon fraction containing hydrocarbons having a carbon chain length above 20. It is noted that some biomass, such as agriculture oils, may produce little or no heavy fraction.

Hydroprocessing also produces a biogenic off gas 115 primarily comprised of carbon dioxide, carbon monoxide, propane, and hydrogen. In some instances, the off gas 115 may also include some methane and ethane. The biogenic off gas 115 is a form of biogas that can be used to produce methanol, as will be described below.

In some embodiments, the off gas 115 is delivered to the optional pre-reforming unit 135 where the ethane, propane, or higher hydrocarbons in the off gas 115 are converted to a lower carbon compound such as methane. In one example, the propane and higher hydrocarbons are converted to methane using steam at a temperature from 500° F. to 1100° F. and in the presence of a nickel-based catalyst, such as nickel chromium. It is contemplated the ethane, propane, or higher hydrocarbons may be converted to methane using any suitable process. The off gas 132 leaving the pre-reforming unit 135 has a higher methane content than the off gas 115 entering the pre-reforming unit 135. In some embodiments, the off gas 115 is delivered to the methane reforming unit 130, such as when a pre-reforming unit 135 is not present.

In some embodiments, the biogenic off gas 115 includes the off gas 126 from the pretreatment unit 125. In some embodiments, the off gas 115 delivered to the pre-reforming unit 135 includes off gas produced from the isomerization process. In some embodiments, one or more of the fractions, such as naphtha, separated during the fractionation process can also be delivered to the pre-reforming unit 135. The pre-reforming unit 135 converts the hydrocarbons in the one or more fractions of the off gas to methane. In some embodiments, the off gas 132 leaving the pre-reforming unit 135 includes methane in an amount from 5 wt. % to 25 wt. % or from 10 wt. % to 20 wt. % based on the total weight of the resulting off gas 132. In addition to methane, the off gas 132 leaving the pre-reforming unit 135 may also include water in an amount from 40 wt. % to 65 wt. %, carbon dioxide in an amount from 20 wt. % to 35 wt. %, and each of hydrogen and carbon monoxide in an amount from 1 wt. % to 5 wt. %, based on the total weight of the resulting off gas 132.

The methane rich off gas 132 from the pre-reforming unit 135 is delivered to a methane reforming unit such as a steam methane reforming unit 130. Thus, the methane rich off gas 132 from the pre-reforming unit 135 is used as the feed gas for the steam methane reforming unit 130. In some embodiments, the off gas 115 from the hydrotreating units 120 is delivered directly to the steam methane reforming unit 130. The off gas 115 may optionally include off gas 126 from the pretreatment unit 125, light ends of the fractionation process such as naphtha, or both. The steam methane reforming unit 130 is configured to convert the methane in the off gas 115, 132 and water to carbon monoxide and hydrogen. The water may be provided from a water source 136 connected to the steam methane reforming unit 130. In particular, the water source 136 can provide water in the form of steam to react with methane to yield hydrogen according to the equation (I):

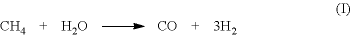

The reaction may occur at a temperature between 700-1100° C. and in the presence of a nickel-based catalyst. In some embodiments, an optional water-gas shift reaction also occurs in the steam methane reforming unit 130 to produce additional hydrogen according to the equation (II):

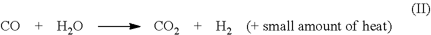

The steam methane reforming unit 130 produces a biogenic syngas 134 that includes hydrogen and at least one of carbon dioxide or carbon monoxide. In some examples, the syngas 134 includes hydrogen, carbon dioxide, and carbon monoxide. In some examples, the syngas 134 includes hydrogen in an amount from 5 wt. % to 15 wt. %, carbon dioxide in an amount from 70 wt. % to 85 wt. %, and carbon monoxide in an amount from 2 wt. % to 12 wt. % based on the total weight of the syngas 134. When the optional water-gas shift reaction is not utilized, the carbon monoxide content in the syngas 134 will be higher. In some embodiments, all of the methane for the steam methane reforming unit 130 are directly or indirectly (via the reforming units 130, 135) provided by the hydrotreating unit 120 such that no additional methane is delivered to the steam methane reforming unit 130. In this respect, the carbon intensity (“CI”) of the fuel generation facility 100 may be lowered. It is noted, however, that during the start up of the steam methane reforming unit 130, some methane may be obtained from an outside source, such as from a natural gas pipeline. Although a steam methane reforming unit 130 is disclosed, it is contemplated that other suitable methane reforming units for producing hydrogen, such as autothermal reforming units or partial oxidation units, may be used.

In one embodiment, a first portion 181 of the syngas 134 is delivered to a hydrogen purification unit such as a pressure swing adsorption (“PSA”) unit 140, and a second portion 182 of the syngas 134 is delivered to the methanol generation unit 150. In some examples, the first portion 181 of the syngas 134 delivered to the PSA unit 140 is an amount from 25% to 90%, from 40% to 75%, or from 55% to 65% of the syngas 134. The second portion 182 of the syngas 134 delivered to the methanol generation unit 150 is an amount from 10% to 75%, from 25% to 60%, or from 35% to 45% of the syngas 134. In some examples, the first portion 181 of the syngas 134 delivered to the PSA unit 140 is an amount from 50% to 65% of the syngas 134, and the second portion 182 of the syngas 134 delivered to the methanol generation unit 150 is an amount from 35% to 50% of the syngas. It is also contemplated that more than 75% of the syngas 134, such as 85% or 100%, can be delivered to either the PSA unit 140 or the methanol generation unit 150. In some examples, the first portion 181 of the syngas 134 undergoes a water-gas shift reaction in the steam methane reforming unit 130 to produce additional hydrogen before delivery to the PSA unit 140. In some examples, the second portion 182 of the syngas 134 is delivered to the methanol generation unit 150 without undergoing the water-gas shift reaction.

The first portion 181 of the syngas 134 is delivered to the PSA unit 140 for purification and separation of the hydrogen. The PSA unit 140 may be any PSA unit suitable for purifying and separating the hydrogen in the syngas 134. In one example, the PSA unit 140 uses beds of solid adsorbents to separate impurities in the syngas 134 from the hydrogen. In this example, the hydrogen is purified and separated from other gases, such as carbon dioxide and carbon monoxide, in the syngas 134. The separated hydrogen forms a hydrogen stream 142 in the PSA unit 140. In some embodiments, the hydrogen stream 142 contains hydrogen in an amount from 90 wt. % to 100 wt. %, at least 95 wt. %, or at least 98 wt. %, based on the total weight of the syngas 134. The beds of solid adsorbents may be regenerated by depressurizing and purging of the beds of the PSA unit 140. The separated hydrogen stream 142 is delivered to the hydrotreating unit 120 which uses the hydrogen to produce the renewable diesel and/or sustainable aviation fuel as discussed above. The separated gases such as carbon dioxide and/or carbon monoxide may be vented. In some embodiments, the separated gases are sent to the methanol generation unit 150 to make more methanol. In some embodiments, the carbon dioxide is isolated from the separated gases, and the carbon dioxide is captured and sequestered, such as in an underground storage unit. Although a PSA unit 140 is disclosed, it is contemplated that other suitable hydrogen purification units, such as membrane units, are also contemplated. In some examples, an amine unit is used to separate out the hydrogen from the syngas 134. For example, the amine unit includes amine-based solvents that can selectively absorb the carbon dioxide from the syngas 134. Then, the amine-based solvents containing the absorbed carbon dioxide are heated to release carbon dioxide. In this manner, the amine-based solvents are regenerated and reused in the absorption process.

The second portion 182 of the syngas 134 is beneficially delivered to the methanol generation unit 150 to produce methanol 155. The methanol generation unit 150 may include any suitable reactors and distillation columns to produce the methanol. Suitable reactors include an adiabatic reactor or an isothermal reactor. In one example, methanol reactors in the methanol generation unit 150 convert the syngas 134 to methanol using a catalytic process involving catalysts such as copper oxide, zinc oxide, or chromium oxide catalysts. In one example, the methanol 155 is produced by the catalytic hydrogenation of carbon monoxide according to the following equation (III):

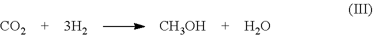

The catalytic process may occur at a pressure from 50 to 100 bar and a temperature from 200° C. to 300° C. In addition to methanol, the catalytic process also produces water. The distillation columns in the methanol generation unit 150 remove the water produced from the catalytic process. In some embodiments, the methanol reactors are configured to deliver any excess hydrogen 152 from the syngas 134 to the PSA unit 140, which may then direct the hydrogen to the hydrotreating unit 120. In some examples, the excess hydrogen 152 forms a part of the excess syngas 134 or purge gas from the methanol reactors that is delivered to the PSA unit 140. In addition to or alternatively, the methanol generation unit 150 may deliver the excess hydrogen directly to the hydrotreating unit 120. In some embodiments, the excess hydrogen 152 is delivered to another hydrogen purification unit such, as a second PSA unit, before delivery to the hydrotreating unit 120. Because the carbon atoms used to produce the methanol (CH₃OH) are biogenic and come from agricultural oils or animal fats, the methanol is considered biomethanol. Therefore, embodiments of present disclosure provide for the production of biomethanol from the waste biogenic off gas 115, 126 that is produced in a renewable diesel or SAF refinery, e.g., produced from the pretreating process, hydroprocessing, or isomerization process. In this respect, the methanol generation unit 150 that produces methanol 155 is integrated with the hydrotreating unit 120 that produces the renewable diesel and/or sustainable aviation fuel.

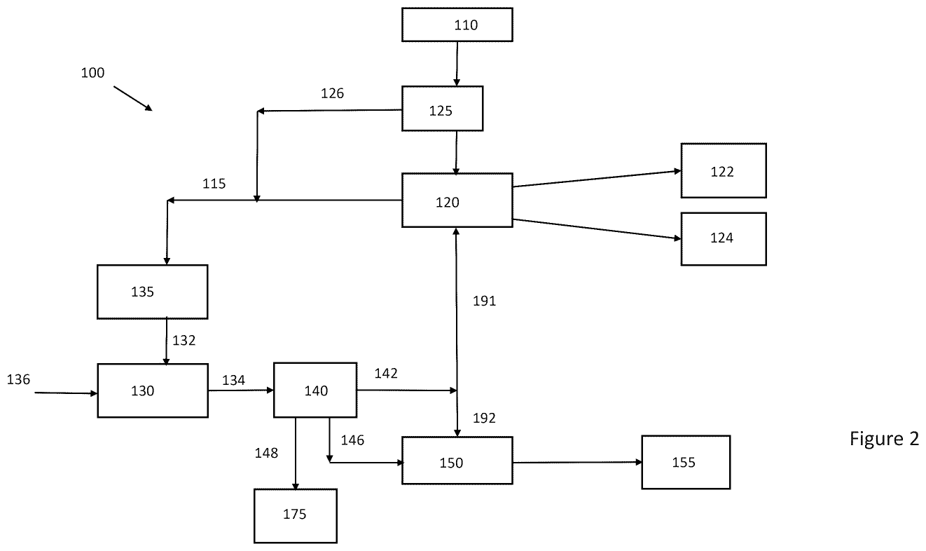

In some embodiments, all of the syngas 134 from the steam methane reforming unit 130 are delivered to the PSA unit 140, as shown in FIG. 2. In this example, the PSA unit 140 receives all of the syngas 134 from the PSA unit 140 and separates the hydrogen from the syngas 134 into a hydrogen stream 142. As shown, a first portion 191 of the separated hydrogen stream 142 from the PSA unit 140 is delivered to the hydrotreating unit 120, and a second portion 192 of the separated hydrogen stream 142 is delivered to the methanol generation unit 150. In some examples, the first portion 191 of the hydrogen stream 142 delivered to the hydrotreating unit 120 is an amount from 25% to 90%, from 40% to 75%, or from 55% to 65% of the hydrogen stream 142. The second portion 192 of the hydrogen stream 142 delivered to the methanol generation unit 150 is an amount from 10% to 75%, from 25% to 60%, or from 35% to 45% of the hydrogen stream 142. In some examples, after removing the hydrogen, a portion or all of the separated gases 146 containing carbon dioxide and carbon monoxide are delivered from the PSA unit 140 to the methanol generation unit 150. The methanol generation unit 150 uses the hydrogen stream 142, the separated gases 146, or both to make methanol 155.

In some examples, at least a portion of the separated gases containing carbon dioxide and carbon monoxide is further processed to remove the carbon dioxide. For example, an amine unit can be used to remove the carbon dioxide from the separated gases. In some examples, the removed carbon dioxide 148 is delivered to a storage unit, such as an underground storage unit 175 for carbon capture and sequestration.

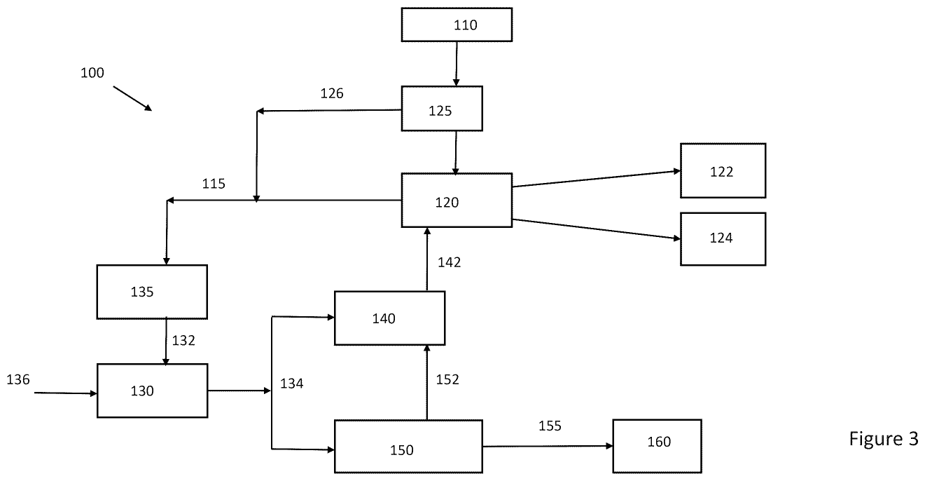

In some embodiments, the facility 100 is configured to make dimethyl ether (“DME”). Referring to FIG. 3, the facility 100 includes a DME generation unit 160 configured to produced DME using the methanol 155 produced by the methanol generation unit 150. In some examples, the DME may be produced using following methanol dehydration reaction (IV):

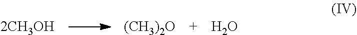

In some examples, the methanol from the methanol generation unit 150 is, optionally, not distilled prior to delivery to the DME generation unit 160 since the DME production process also produces water that requires removal. In some embodiments, the methanol generation unit 150 may include a reactor configured to produce both methanol and DME such that the DME generation unit 160 is optional.

Embodiments of the present disclosure beneficially provides methods of producing biofuels at a surprisingly low carbon intensity (“CI”). It is believed the present disclosure allows for the production of biofuel such as biomethanol at a lower capital cost and a lower operating cost. The capital cost is lower due to the sharing of equipment at the biofuel generation facility to co-generate methanol and RD/SAF. Examples of shared equipment include the SMR 130, as well as the control room, logistic infrastructure, and other ancillary equipment. The operating cost is lower because the biogenic off gas advantageously allows for the production of more gallons of renewable fuels per unit of feedstock, thereby increasing efficiency.

### Example

In one example, the feedstock includes distillers corn oil, beef tallow, and used cooking oil. The feedstock is supplied at 10 k barrels/day (155 k tons/day). The feedstock is hydrotreated to produce renewable diesel or sustainable aviation fuel (SAF) at around 9-9.5 k bpd. The yield obtained is about 90-95% of the feedstock input. The amount of off gas sent to the steam methane reforming unit (SMR) is around 290-350 tons/day (7.4-8.7 MMSCFD). In this example, the makeup of the off gas includes CO₂ at 23.1%, CO at 17.6%, H₂ at 1.2%, methane at 4%, ethane at 7.5%, propane at 9.8%, and butane at 6.7% of the total mass of the off gas. The SMR produces a syngas having 9.7% H₂, 78% CO₂, and 7% CO. Approximately 42% of the syngas is sent to the methanol generation unit, and 58% of the syngas is sent to the PSA unit. The PSA produces 13 MMSCFD (65 tons/day) of hydrogen. The methanol generation unit produces 137 tons/day (41650 gal/day) of biomethanol. Also, 1.5 MMSCFD (34.3 tons/day) of hydrogen from the methanol generation unit is recycled to the PSA unit.

In one embodiment, a method of producing methanol includes supplying biomass feedstock to a hydrotreating unit to produce a biofuel and a biogenic off gas. The biogenic off gas is delivered to a methane reforming unit to generate a syngas having higher hydrogen content than the biogenic off gas. The method also includes delivering a first portion of the syngas to a hydrogen purification unit to produce a hydrogen stream. The hydrogen stream is delivered to the hydrotreating unit. A second portion of the syngas is delivered to a methanol generation unit. Methanol is generated from the second portion of the syngas by the methanol generation unit.

In another embodiment, a method of producing biofuels includes supplying biomass feedstock to a hydrotreating unit to produce a first biofuel and a first biogenic off gas. The first biogenic off gas is delivered to a pre-reforming unit to produce a second biogenic off gas having a higher methane content than the first biogenic off gas. The second biogenic off gas is delivered to a methane reforming unit to generate a syngas having higher hydrogen content than the second biogenic off gas. A first portion of the syngas is delivered to a pressure swing unit. A second portion of the syngas is delivered to a methanol generation unit, and a second biofuel is generated from the second portion of the syngas. Hydrogen from the methanol generation unit is delivered to the pressure swing unit or the hydrotreating unit. In some examples, the hydrogen is a part of the excess syngas or purge gas delivered from the methanol generation unit.

In another embodiment, a method of producing biofuels includes supplying biomass feedstock to a hydrotreating unit to produce a first biofuel and a first biogenic off gas. The method also includes pre-reforming the first biogenic off gas to produce a second biogenic off gas having a higher methane content than the first biogenic off gas. The second biogenic off gas is reformed to generate a syngas having a higher hydrogen content than the second biogenic off gas. Hydrogen is separated from a first portion of the syngas using a hydrogen purification unit. The separated hydrogen is delivered to the hydrotreating unit. A second portion of the syngas is used to generate a second biofuel.

In another embodiment, a method of producing methanol includes supplying biomass feedstock to a hydrotreating unit to produce a biofuel and a biogenic off gas. The method also includes delivering the biogenic off gas to a methane reforming unit to generate a syngas having higher hydrogen content than the biogenic off gas. The syngas is delivered to a hydrogen purification unit to produce a hydrogen stream and a gas stream. In some examples, the gas stream includes carbon dioxide and carbon monoxide. A first portion of the hydrogen stream is delivered to the hydrotreating unit, and a second portion of the hydrogen stream is delivered to a methanol generation unit. Methanol is generated using the methanol generation unit.

In one or more of the embodiments described herein, the biofuel is at least one of a renewable diesel or a sustainable aviation fuel.

In one or more of the embodiments described herein, the method includes delivering the biogenic off gas to a pre-reforming unit prior to being delivered to the methane reforming unit.

In one or more of the embodiments described herein, the biogenic off gas is a first biogenic off gas, and the pre-reforming unit converts the first biogenic off gas into a second biogenic off gas that has a higher methane content.

In one or more of the embodiments described herein, delivering the biogenic off gas to a methane reforming unit comprises delivering the second biogenic off gas to the methane reforming unit.

In one or more of the embodiments described herein, the first portion of the syngas is an amount from 40% to 75% of the syngas.

In one or more of the embodiments described herein, the first portion of the syngas is an amount from 50% to 65% of the syngas.

In one or more of the embodiments described herein, the second portion of the syngas is an amount from 25% to 60% of the syngas.

In one or more of the embodiments described herein, the second portion of the syngas is an amount from 35% to 50% of the syngas.

In one or more of the embodiments described herein, the hydrogen purification unit comprises a pressure swing unit configured to separate hydrogen from the first portion of syngas to form the hydrogen stream.

In one or more of the embodiments described herein, the method includes delivering hydrogen from the methanol generating unit to the pressure swing unit or the hydrotreating unit.

In one or more of the embodiments described herein, the biogenic off gas includes propane, carbon dioxide, and hydrogen.

In one or more of the embodiments described herein, the biomass feedstock comprises triglycerides, fatty acids, or combinations thereof.

In one or more of the embodiments described herein, the method includes delivering naphtha from the hydrotreating unit to a pre-reforming unit prior to being delivered to the methane reforming unit.

In one or more of the embodiments described herein, the second biofuel is at least one of methanol or dimethyl ether.

In one or more of the embodiments described herein, the first portion of the syngas is an amount from 40% to 75% of the syngas.

In one or more of the embodiments described herein, the second portion of the syngas is an amount from 25% to 60% of the syngas.

In one or more of the embodiments described herein, the method includes separating hydrogen in the first portion of the syngas using the pressure swing unit.

In one or more of the embodiments described herein, the method includes delivering the separated hydrogen to the hydrotreating unit.

In one or more of the embodiments described herein, the method includes pretreating the biomass feedstock prior to delivering the biomass feedstock to the hydrotreating unit.

In one or more of the embodiments described herein, pretreating the biomass feedstock comprises removing one or more impurities in the biomass feedstock.

In one or more of the embodiments described herein, pretreating the biomass feedstock produces a third biogenic off gas, and the method further comprises delivering the third biogenic off gas to the pre-reforming unit.

In one or more of the embodiments described herein, the method includes delivering a portion of the gas stream to the methanol generating unit.

In one or more of the embodiments described herein, the method includes separating carbon dioxide from the gas stream.

In one or more of the embodiments described herein, the method includes delivering the carbon dioxide to a storage unit or sequestration.

In one or more of the embodiments described herein, the hydrogen purification unit is a pressure swing adsorption unit or an amine unit.

In one or more of the embodiments described herein, the methane reforming unit is a steam methane reforming unit or an autothermal methane reforming unit.

Although the present disclosure and its advantages have been described in detail, it should be understood that various changes, substitutions and alterations may be made herein without departing from the spirit and scope of the invention as defined by the appended claims.

1. A method of producing methanol, comprising:
supplying biomass feedstock to a hydrotreating unit to produce a biofuel and a biogenic off gas;
delivering the biogenic off gas to a methane reforming unit to generate a syngas having higher hydrogen content than the biogenic off gas;
delivering a first portion of the syngas to a hydrogen purification unit to produce a hydrogen stream;
delivering the hydrogen stream to the hydrotreating unit;
delivering a second portion of the syngas to a methanol generation unit; and
generating methanol from the second portion of the syngas.

2. The method of claim 1, wherein the biofuel is at least one of a renewable diesel or a sustainable aviation fuel.

3. The method of claim 1, further comprising delivering the biogenic off gas to a pre-reforming unit prior to being delivered to the methane reforming unit.

4. The method of claim 3, wherein the biogenic off gas is a first biogenic off gas, and the pre-reforming unit converts the first biogenic off gas into a second biogenic off gas that has a higher methane content than the first biogenic off gas.

5. The method of claim 4, wherein delivering the biogenic off gas to a methane reforming unit comprises delivering the second biogenic off gas to the methane reforming unit.

6. The method of claim 1, wherein the first portion of the syngas is an amount from 40% to 75% of the syngas.

7. The method of claim 1, wherein the first portion of the syngas is an amount from 50% to 65% of the syngas.

8. The method of claim 1, wherein biogenic off gas includes carbon monoxide, propane, carbon dioxide, and hydrogen.

9. The method of claim 1, further comprising delivering naphtha from the hydrotreating unit to a pre-reforming unit prior to being delivered to the methane reforming unit.

10. The method of claim 1, wherein the hydrogen purification unit comprises a pressure swing unit configured to separate hydrogen from the first portion of syngas to form the hydrogen stream.

11. The method of claim 10, further comprising delivering hydrogen from the methanol generating unit to the pressure swing unit or the hydrotreating unit.

12. A method of producing biofuels, comprising:
supplying biomass feedstock to a hydrotreating unit to produce a first biofuel and a first biogenic off gas;
delivering the first biogenic off gas to a pre-reforming unit to produce a second biogenic off gas having a higher methane content than the first biogenic off gas;
delivering the second biogenic off gas to a methane reforming unit to generate a syngas having higher hydrogen content than the second biogenic off gas;
delivering a first portion of the syngas to a pressure swing unit;
delivering a second portion of the syngas to a methanol generation unit;
generating a second biofuel from the second portion of the syngas; and
delivering hydrogen from the methanol generation unit to the pressure swing unit or the hydrotreating unit.

13. The method of claim 12, wherein the second biofuel is at least one of methanol or dimethyl ether.

14. The method of claim 12, wherein the first portion of the syngas is an amount from 40% to 75% of the syngas.

15. The method of claim 12, wherein the second portion of the syngas is an amount from 25% to 60% of the syngas.

16. The method of claim 12, further comprising separating hydrogen in the first portion of the syngas using the pressure swing unit.

17. The method of claim 16, further comprising delivering the separated hydrogen to the hydrotreating unit.

18. The method of claim 12, further comprising pretreating the biomass feedstock prior to delivering the biomass feedstock to the hydrotreating unit.

19. A method of producing methanol, comprising:
supplying biomass feedstock to a hydrotreating unit to produce a biofuel and a biogenic off gas;
delivering the biogenic off gas to a methane reforming unit to generate a syngas having higher hydrogen content than the biogenic off gas;
delivering the syngas to a hydrogen purification unit to produce a hydrogen stream and a gas stream;
delivering a first portion of the hydrogen stream to the hydrotreating unit;
delivering a second portion of the hydrogen stream to a methanol generation unit; and
generating methanol using the methanol generation unit.

20. The method of claim 19, further comprising delivering a portion of the gas stream to the methanol generating unit.
